(19) United States
(12) Patent Application Publication
(10) Pub. No.: US 2021/0085443 A1
(43) Pub. Date: Mar. 25, 2021

(54) PRE-SHAPED ALLOGRAFT IMPLANT FOR RECONSTRUCTIVE SURGICAL USE AND METHODS OF MANUFACTURE AND USE

(71) Applicant: Allosource, Centennial, CO (US)

(72) Inventors: Ergun Kocak, Gahanna, OH (US); Lauren Blume, Highlands Ranch, CO (US); Jeffrey Chiesa, Denver, CO (US); Kenneth Blood, Littleton, CO (US); Reginald Stilwell, Parker, CO (US)

(21) Appl. No.: 16/707,681

(22) Filed: Dec. 9, 2019

Related U.S. Application Data
(60) Provisional application No. 62/905,485, filed on Sep. 25, 2019.

(51) Int. Cl. A61F 2/00; A61L 27/36; A61F 2/12; B65B 5/04; B65B 55/02

(52) U.S. Cl. CPC A61F 2/0095; A61L 27/3691; A61L 27/362; A61F 2/12; B65B 5/045; A61L 2430/40; A61L 2300/404; A61L 2300/104; A61L 2300/206; A61L 2300/252; A61L 2430/04; B65B 55/02

(57) ABSTRACT

An acellular dermal matrix (ADM) graft product includes an ADM graft derived from full-thickness skin, with a pre-formed shape having a mesh pattern formed therein. The ADM graft is disposed in a sterilization package and irradiated to provide a two year shelf-life for the ADM graft product, which is used in the time-saving, efficient, and effective surgical reconstruction of soft tissue defects. One manufacturing method involves providing a portion of full-thickness donor-derived skin, removing an epidermis and a fat layer form the portion of the skin, decellularizing the portion of the skin to form a portion of ADM graft material, pre-shaping and meshing the ADM graft material, verifying a thickness of the ADM graft material, and packaging the pre-shaped, meshed ADM graft material in a sterilizable package and irradiating for a two-year shelf-life to form the ADM graft product. Other embodiments are also disclosed.

### REFERENCE TO PRIOR PATENT APPLICATION

This application claims the benefit under 35 U.S.C. 119 (e) of U.S. Provisional Patent Application No. 62/905,485, filed Sep. 25, 2019 by Ergun Kocak, Lauren Blume, Kenneth Blood, Reginald Stilwell, and Jeffrey Chiesa for “PRE-SHAPED ALLOGRAFT IMPLANT FOR RECONSTRUCTIVE SURGICAL USE AND METHODS OF MANUFACTURE AND USE,” all of which patent application is hereby incorporated herein by reference.

### BACKGROUND

An allograft includes bone, tendon, skin, or other types of tissue that is transplanted from one person to another. Allografts are used in a variety of medical treatments, such as knee replacements, bone grafts, spinal fusions, eye surgery, and skin grafts for reconstructive surgery and for the severely burned. Allografts come from voluntarily donated human tissue obtained from cadaveric donor-derived, living-related, or living-unrelated donors and can help patients regain mobility, restore function, enjoy a better quality of life, and even save lives in the case of cardiovascular tissue or skin.

An acellular dermal matrix (ADM) graft is a soft connective tissue graft generated by a decellularization process that preserves the intact extracellular skin matrix. Upon implantation, the ADM structure serves as a scaffold for donor-side cells to facilitate subsequent incorporation and revascularization. ADMs are manufactured utilizing known methods of decellularization by means of ionic and nonionic detergent methods, as well as those utilizing enzymatic processes and other techniques such as those listed in “Decellularization of Tissues and Organs,” Gilbert, et al, 2006 (https://www.ncbi.nlm.nih.gov/pubmed/16519932).

Currently, ADM grafts are primarily derived from decellularized cadaveric skin and must be shaped and/or cut as necessary by the surgeon either prior to or during a surgical procedure. Such grafts are also commonly formed from solid or perforated ADM. As a result, existing ADM grafts present efficiency, efficacy, and repeatability challenges when used for reconstructive surgery purposes.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

One embodiment provides a method of manufacturing an acellular dermal matrix (ADM) graft product for use in a reconstructive surgical procedure. The method may include the following steps: (1) providing a portion of donor-derived skin, the portion of the donor-derived skin having a full thickness; (2) removing an epidermis layer and a fat layer from the portion of the donor-derived skin to form a portion of dermal tissue; (3) decellularizing the portion of the dermal tissue to form a portion of ADM graft material; (4) forming the portion of the ADM graft material into a pre-defined shape in anticipation of the reconstructive surgical procedure; (5) fenestrating the pre-defined shape into a mesh pattern; (6) verifying that a thickness of the pre-defined shape equals a specified thickness; (7) packaging the pre-defined shape in a medical sterilization pouch to form a packaged, pre-shaped, and meshed ADM graft; and (8) irradiating the packaged, pre-shaped, and meshed ADM graft to a sterility assurance level of 10⁻⁶ to form the ADM graft product.

Another embodiment provides a pre-shaped, meshed acellular dermal matrix (ADM) graft stored as a packaged graft product prepared by a process comprising the steps of: (1) providing a portion of ADM tissue having a thickness between 1 mm and 2 mm; (2) fenestrating the portion of the ADM tissue in a mesh pattern extending over an entirety of the portion of the ADM tissue; (3) scoring the portion of the ADM tissue into a pre-defined shape to form the pre-shaped, meshed ADM graft; (4) verifying the thickness of the pre-shaped, meshed ADM graft; (5) packaging the pre-shaped, meshed ADM graft in a medical sterilization pouch; and (6) irradiating the pre-shaped, meshed ADM graft within the medical sterilization pouch to a sterility assurance level of 10⁻⁶ to form the packaged graft product.

Yet another embodiment provides an acellular dermal matrix (ADM) graft product. The ADM graft product may include an ADM graft derived from full-thickness skin, the ADM graft having a pre-formed shape with a mesh pattern formed therein, as well as a medical sterilization pouch sealed about the ADM graft, wherein when the medical sterilization pouch and the ADM graft are irradiated to a sterility assurance level of 10⁻⁶, the ADM graft product has a shelf-life of two years.

Other embodiments provide an ADM graft that combines the ADM as designed with antimicrobial elements that mitigate or prevent complications arising from post-surgical infections. Antimicrobial agents that are compatible with the ADM include silver in its colloidal, elemental or ionic form. The silver may be complexed with chelating agents or may be added directly to the ADM prior to final packaging. Similarly other antimicrobial agents may be combined with the ADM. Other agents well known to be used medically are chlorhexidine gluconate and antimicrobial peptides of various amino acid chain length.

Other embodiments are also disclosed.

Additional objects, advantages and novel features of the technology will be set forth in part in the description which follows, and in part will become more apparent to those skilled in the art upon examination of the following, or may be learned from practice of the technology.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIGS. 1A-1B illustrate respective front-plan and perspective views of one embodiment of a pre-shaped, meshed acellular dermal matrix (ADM) graft derived from full-thickness skin;

FIGS. 2A-2B illustrate respective top-perspective and bottom-perspective views of one embodiment of scoring tool for manufacturing the pre-shaped, meshed ADM graft of FIGS. 1A-1B;

FIGS. 3A-3B illustrate front-plan views of an exemplary mesh, or fenestration, pattern of the pre-shaped, meshed ADM graft of FIGS. 1A-1B, shown in an open position and in a resting position, respectively;

FIG. 4 illustrates a perspective view of an exemplary skin mesher for forming the mesh pattern of FIGS. 3A-3B;

FIG. 5 illustrates a first perforated prior art ADM graft for comparison to the pre-shaped, meshed ADM graft of FIGS. 1A-1B;

FIG. 6 illustrates a second perforated prior art ADM graft for comparison to the pre-shaped, meshed ADM graft of FIGS. 1A-1B;

FIGS. 7A-7B illustrate perspective views of a fluid egress testing device in respective first and second stages of fluid egress testing of the pre-shaped, meshed ADM graft of FIGS. 1A-1B, the first perforated ADM graft of FIG. 5, and the second perforated ADM graft of FIG. 6;

FIGS. 8A-8B provide a table reflecting multiple sets of drainage time measurements captured during the fluid egress testing performed using the testing device of FIGS. 7A-7B;

FIG. 9 provides a summary graft of the drainage time measurements shown in FIGS. 8A-8B;

FIG. 10 illustrates a front perspective view of an ADM graft pocket formed by joining two of the pre-shaped, meshed ADM grafts of FIGS. 1A-1B together;

FIG. 11 illustrates a front view of a pre-shaped, meshed ADM graft product in which the pre-shaped, meshed ADM graft of FIGS. 1A-2A is packaged for storage in a sealed medical sterilization pouch;

FIG. 12 illustrates the pre-shaped, meshed ADM graft product of FIG. 11 further packaged in a medical peel pouch; and

FIG. 13 provides a flowchart depicting an exemplary method of manufacturing an embodiment of the pre-shaped, meshed ADM graft of FIGS. 1A-1B and the packaged ADM graft product of FIGS. 11-12.

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

Various embodiments of the products and associated methods of manufacture and use described herein relate to a pre-shaped, meshed or fenestrated acellular dermal matrix (ADM) graft derived from full-thickness human, donor-derived skin for use in the surgical reconstruction of soft tissue defects resulting from trauma, disease, or surgical intervention. For example, embodiments of the ADM graft discussed herein may be used in the surgical specialty of plastic surgery, and particularly in prepectoral and post-mastectomy breast reconstruction, where the ADM graft is an adjunct to integumental repair of the surgical site.

Embodiments of the ADM graft may be packaged and irradiated for long-term sterile storage in a manner that allows them to be used in surgical procedures within two years of packaging. In use, embodiments of the pre-shaped, meshed ADM graft provide the surgeon with a mechanism to restore function to and support integumental tissue after surgical intervention in a manner that is repeatable, effective, and time efficient by leveraging a manufactured, pre-shaped and meshed ADM graft product that is derived from full-thickness skin. Embodiments of the ADM graft facilitate fluid drainage from the surgical site to discourage seroma formation, increase the rate of integration of the ADM graft with the body, and provide a reliable, repeatable solution the surgeon may use “off the shelf” rather than utilizing valuable time and resources for graft processing in preparation for or during the surgical procedure.

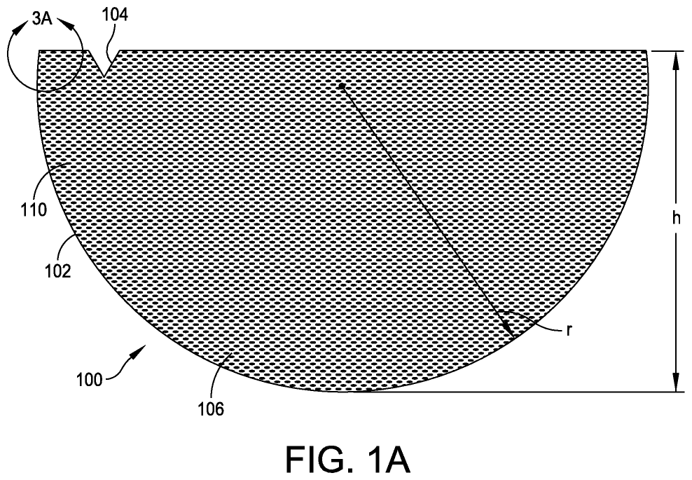

Turning to exemplary embodiments, FIGS. 1A-1B illustrate respective front-plan and perspective views of one embodiment of a pre-shaped, meshed ADM graft 100 derived from decellularized, full-thickness skin. Using full-thickness skin as the source for the ADM graft 100 ensures that the ADM graft 100 has sufficient biomechanical properties to support varying surgical requirements, including, for example, a suitable ultimate tensile strength, suture pull-out resistance, and a Young's modulus indicative of a soft and supple graft.

In this embodiment, the pre-shaped, meshed ADM graft 100 may have a pre-formed shape approximating a circle with a portion of the top removed (i.e., slightly larger than a semi-circle). In one embodiment, as detailed in FIGS. 1A-1B, the pre-shaped ADM graft may form a generally semi-circular tissue portion 102 having a radius, r, of 9 cm. The semi-circular tissue portion 102 may approximate a circle having a top portion of the circle removed in a straight line disposed perpendicular to the radius, r, of the circle. The tissue portion 102 may have a total height, h, of 10 cm, and a material thickness, t, of 1.0-2.0 mm. Additional pre-shaped ADM graft embodiments may feature various circular or elliptical shapes with diameters ranging from 10 cm to 22 cm. The circular or elliptical tissue portion of the ADM graft may feature a removed top portion, as shown in FIGS. 1A-1B, or an in-tact top portion, as necessary or desired for the intended surgical preparation.

In addition, the pre-shaped, meshed ADM graft 100 may include a notch 104 to indicate which surface provides a basement membrane surface 106, or the dermal surface to be implanted towards the patient's vascular bed. In one embodiment, as shown in FIGS. 1A-1B, the notch 104 of the graft 100 may be disposed in the top left corner to indicate the basement membrane surface 106. In other embodiments, the basement membrane may be removed.

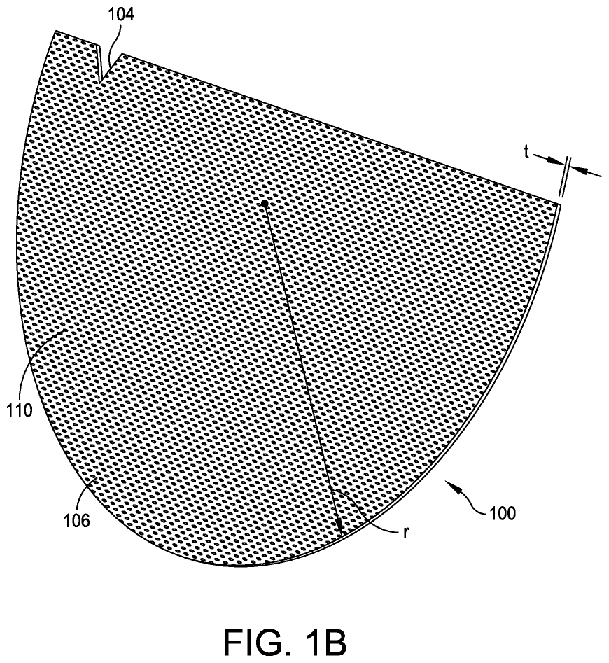

The decellularized, full-thickness dermal tissue may be shaped and cut into the pre-shaped ADM graft 100 using an appropriately designed scoring tool along with a cutting tool such as, for example, a surgical scalpel or a surgical scissor. FIGS. 2A-2B illustrate respective front and rear perspective views of one embodiment of a scoring tool 130 featuring a semi-circular edge pattern 132 that incorporates a raised notch 134 configured to form the indicator notch 104 in the pre-shaped ADM graft 100. To manufacture the pre-shaped ADM graft 100, an embodiment of the scoring tool 130 may be placed upon a portion of full-thickness dermal tissue and used to “stamp” out the notched, semi-circular tissue portion 102 from a larger ADM tissue portion. The cutting tool (not shown) may be used to trim excess tissue from around a perimeter of the scoring tool 130.

The pre-shaped nature of the ADM graft 100 disclosed herein saves the surgeon valuable time during a surgical procedure because there is no (or minimal) need for the surgeon to shape, cut, or otherwise form the ADM graft into a desired shape during surgical preparation. Instead, the surgeon may simply select an appropriately pre-shaped ADM graft for the particular surgery and proceed.

Embodiments of the pre-shaped ADM graft 100 may additionally include a mesh or fenestration pattern to allow for increased fluid flow through the graft 100, thereby reducing the chances of post-surgical seroma formation, a frequent complication after surgeries using existing ADM grafts. Pre-meshing also prevents the surgeon from having to perform any type or kind of meshing procedures during surgical preparation or during a surgical procedure and ensures an optimal mesh ratio to provide maximum fluid egress, or drainage, from the surgical site to prevent seroma formation and a maximum graft surface area for improved integration into the body post procedure.

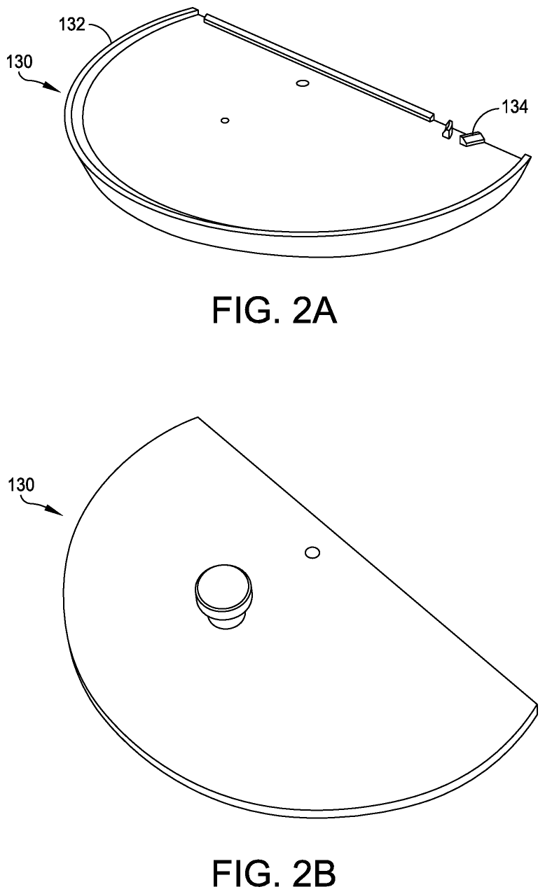

FIGS. 3A-3B illustrate respective front views of an exemplary mesh, or fenestration, pattern 108 applied to the pre-shaped, meshed ADM graft 100, shown in an open position, A, in which the mesh pattern appears as a series of holes 110 (FIG. 3A) and in a resting position, B, in which the mesh pattern 108 appears as a series of straight slits or lines 112 (FIG. 3B). In this embodiment, the mesh pattern 108 may feature a 1:1 graft:space ratio in which each mesh hole 110/line 112 has a length, L, of 1.5 mm, an end-to-end offset, E_{O}, of 1.5 mm, and a lateral offset, L_{O}, of 1 mm. Alternative embodiments may feature a different mesh ratio and/or any appropriate and/or desired material and line dimensions. For example, in one embodiment the mesh pattern 108 may feature a 2:1 graft:space ratio, with a material thickness of 0.8-2.5 mm.

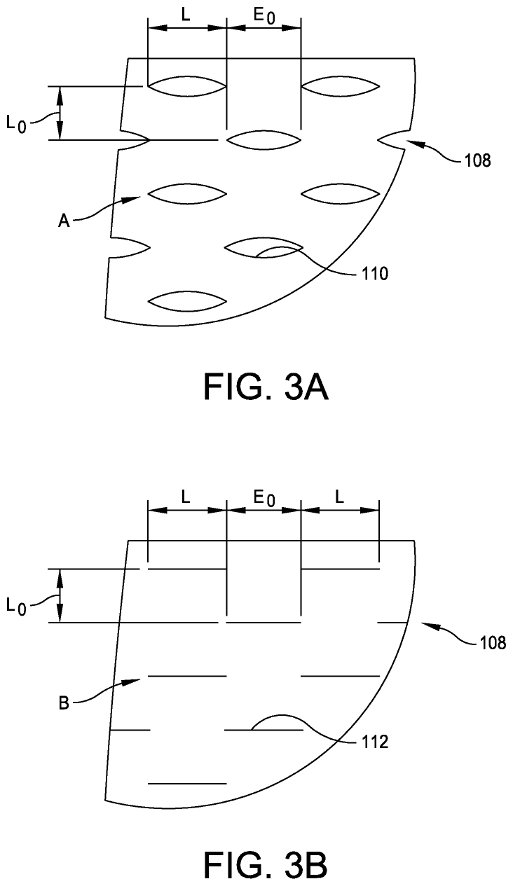

The mesh or fenestration pattern 108 may be formed in the pre-shaped, meshed ADM graft 100 using a standard “skin mesher” 140 such as, for example, a 4MED (or Rosenberg) Skin Graft Mesher (Distributed by Exsurco Medical, Wakeman, Ohio). As shown in FIG. 4, a portion of decellularized, full-thickness dermal tissue 101 or, alternatively, a pre-shaped semi-circular tissue portion 102 may be inserted into the skin mesher 140, which has been adjusted to the appropriate mesh or fenestration settings, for application of the mesh pattern 108 to the tissue 101.

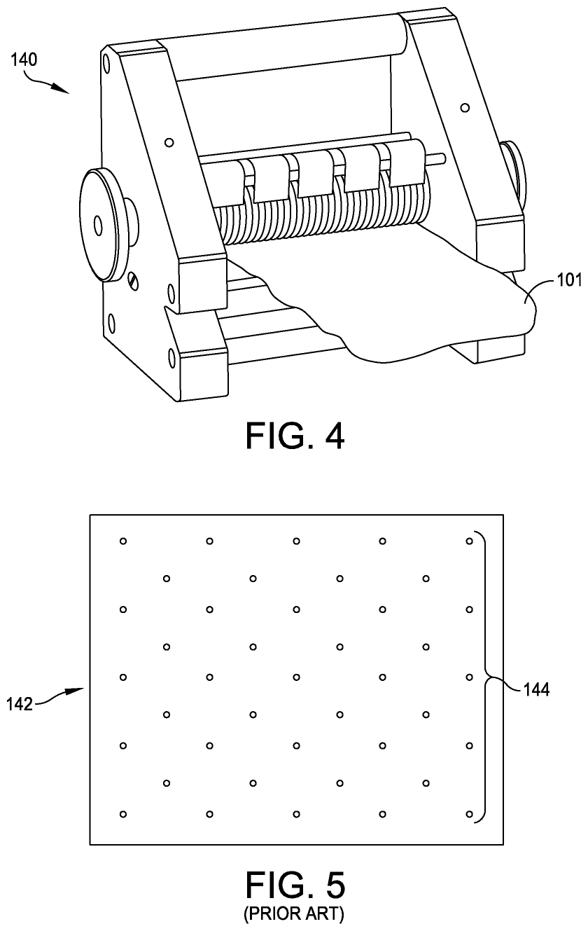

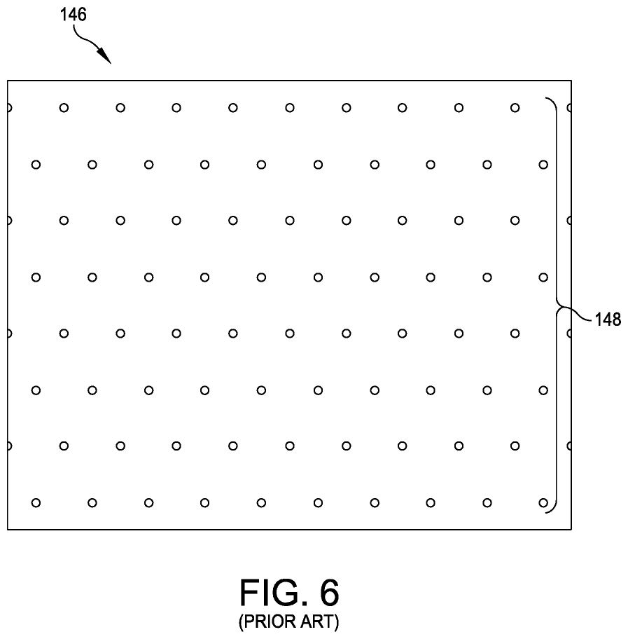

A fluid egress study was completed to exhibit the increased fluid egress, or drainage, properties of the pre-shaped, meshed ADM graft 100. In the study, the fluid drainage properties of the pre-shaped, meshed ADM graft 100 were compared to those of a prior art first perforated ADM graft 142, shown in FIG. 5, having a first perforation density pattern 144 of 41 perforations per 320 cm², or approximately 0.128 perforations per cm², and a prior art second perforated ADM graft 146, shown in FIG. 6, having a second perforation density pattern 148 of 80 perforations per 320 cm², or approximately 0.25 perforations per cm² and approximately twice that of the first perforation density pattern 144. Three samples of each were tested, each sample having a thickness between 0.9-2.0 mm.

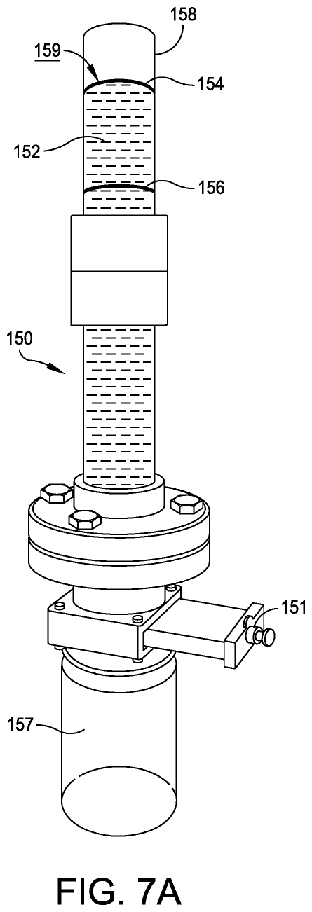

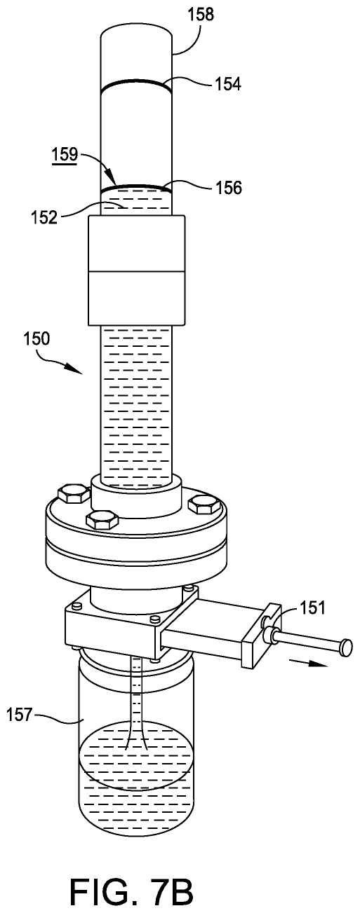

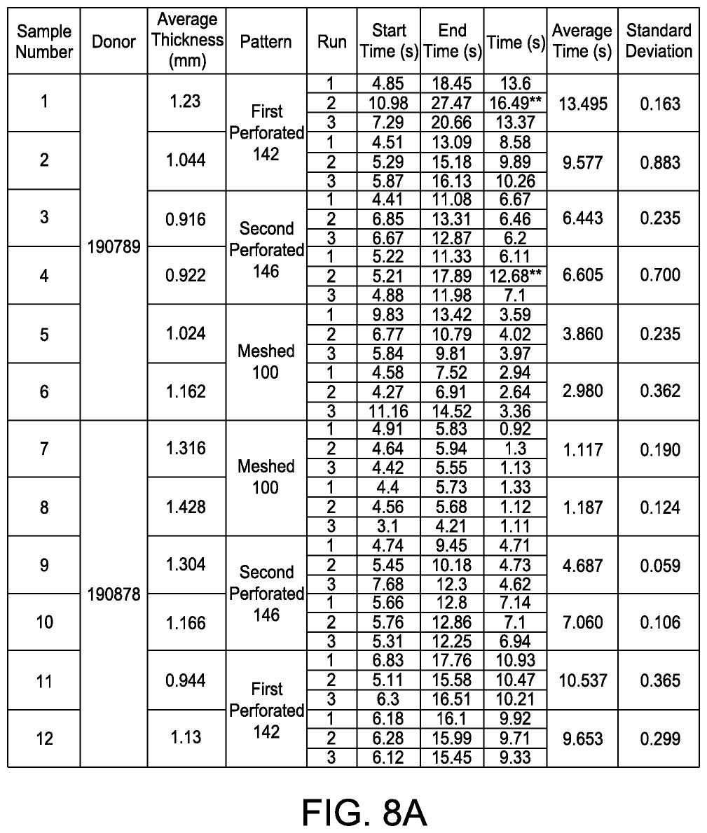

FIGS. 7A-7B illustrate perspective views of a fluid egress testing device 150 in first and second stages of egress testing, respectively. Upon release of a valve 151, a fluid 152 was passed from a fluid column 158, through the respective tested ADM graft (i.e., the pre-shaped meshed ADM graft 100, the first perforated ADM graft 142, or the second perforated ADM graft 144) stretched across the fluid column 158 (not shown) and into a waste container 157. An egress or drainage-time measurement was taken of the time required for a top surface 159 the fluid 152 to fall 8.5 inches from a first fluid-level line 154 to a second fluid-level line 156 along the fluid column 158 of the fluid egress testing device 150, as shown in FIGS. 7A-7B, respectively. The drainage time for the fluid surface 159 to pass from the first line 154 to the second line 156 was measured in triplicate for each of the pre-shaped meshed ADM graft 100, the first perforated ADM graft 142, and the second perforated ADM graft 146. The drainage time measurements are provided in the table of FIGS. 8A-8B. As summarized in the chart of FIG. 9, the fluid egress study showed that the pre-shaped, meshed ADM graft 100 having the 1:1 graft:space ratio demonstrated significantly improved fluid egress properties, namely approximately 3× and 5× faster fluid egress as compared to the first and the second perforation density patterns 144, 148 of the first and the second perforated grafts 142, 146, respectively.

As discussed above, the mesh pattern 108 also increases the surface area of the pre-shaped, meshed ADM graft 100, which, in turn, abets a rate of integration of the graft 100 during the healing process after surgical intervention. The surface area calculations below compare the pre-shaped, meshed ADM graft 100 with the first and the second perforated grafts 142, 146 having the first and the second perforation patterns 144, 148, respectively, discussed above in relation to FIGS. 5-6. In summary, the surface area of a 2×2 cm² meshed ADM graft having a 1 mm thickness and 130, 1.5 mm long mesh lines provides a 97.5% increase in surface area over a 2×2 cm² solid, non-meshed ADM graft, as shown below:

$$Surface Area = ( area of top of graft ) + ( \# mesh lines ) ( perimeter of mesh hole ) ( thickness )$$

$$Surface Area = ( 4 cm ) + ( 130 ) ( 2 * 1.5 mm ) ( 1 mm )$$

$$Surface Area = 4 cm + 3.9 cm = 7.9 cm$$

$$Original Solid Graft Area = ( 2 cm ) ( 2 cm ) = 4 {cm}^{2}$$

$$Increase in Surface Area from Meshing = \frac{7.9 - 4}{4} * 100 \% = 97.5 \%$$

The first perforated graft 142 having a 16 cm×20 cm perimeter and a 1 mm thickness, with a perforation density pattern 144 of 41 perforations per a 320 cm² area, each perforation having a 0.15 cm radius, provides only a 0.3% surface-area increase over a 16 cm×20 cm solid, non-meshed ADM graft, as shown below:

$$Surface Area = ( area of top of graft ) + ( \# holes ) ( surface area of inside of hole )$$

$$Surface Area = ( 320 {cm}^{2} - ( 41 ) ( π * {.15}^{2} ) ) + ( 41 ) ( 2 * π * .15 cm ) ( .1 cm )$$

$$Surface Area = 317.10188 {cm}^{2} + 3.86384 {cm}^{2} = 320.966 {cm}^{2}$$

$$Original Solid Graft Area = ( 16 cm ) ( 20 cm ) = 320 {cm}^{2}$$

$$Increase in Surface Area from Perforating = \frac{320.966 - 320}{320} * 100 \% = 0.3 \%$$

The second perforated graft 146 having a 16 cm×20 cm perimeter and a 1 mm thickness, with a perforation density pattern 148 of 80 perforations per a 320 cm² area, each perforation having a 0.15 cm radius, provides only a 0.59% surface-area increase over a 16 cm×20 cm solid, non-meshed ADM graft, as shown below:

$$Surface Area = ( area of top of graft ) + ( \# holes ) ( surface area of inside of hole )$$

$$Surface Area = ( 320 {cm}^{2} - ( 80 ) ( π * {.15}^{2} ) ) + ( 80 ) ( 2 * π * .15 cm ) ( .1 cm )$$

$$Surface Area = 314.34513 {cm}^{2} + 7.5392 {cm}^{2} = 321.884 {cm}^{2}$$

$$Original Solid Graft Area = ( 16 cm ) ( 20 cm ) = 320 cm$$

$$Increase in Surface Area from Perforating = \frac{321.884 - 320}{320} * 100 \% = 0.59 \%$$

Thus, the fenestration pattern 108 applied to the pre-shaped, meshed ADM graft 100 significantly increases the exposed surface area of the graft over both existing solid and perforated grafts. This increase causes the pre-shaped, meshed ADM graft 100 to integrate into the human body much more rapidly during the healing process after surgical intervention.

In one embodiment, the pre-shaped, meshed ADM graft 100 may be formed of the ADM derived from full-thickness skin, as discussed above, combined with antimicrobial elements that mitigate or prevent complications arising from post-surgical infections. Antimicrobial agents compatible with the ADM may include, for example, silver in its colloidal, elemental, or ionic form. The silver may be complexed with chelating agents or may be added directly to the ADM prior to final packaging. Similarly, other antimicrobial agents may be combined with the ADM. Other agents known to be used medically may include chlorhexidine gluconate and antimicrobial peptides having various amino acid chain lengths.

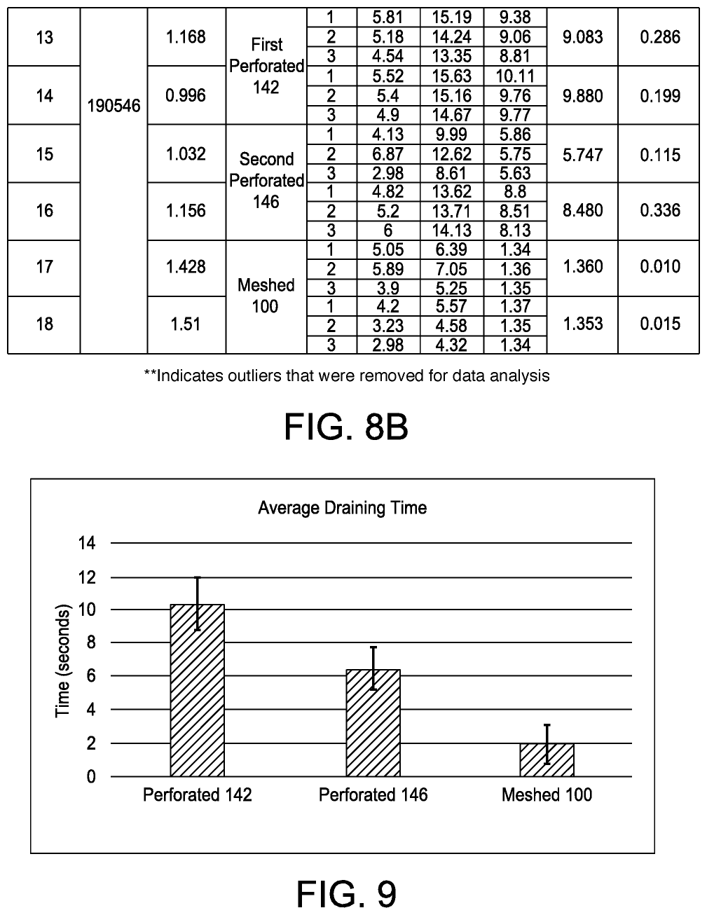

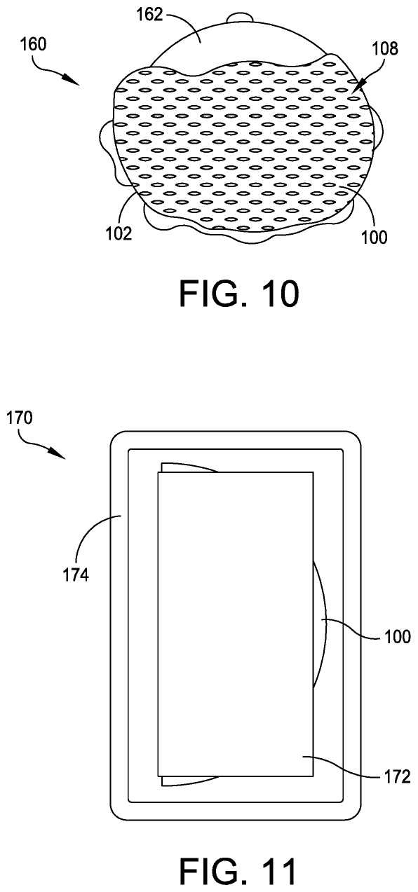

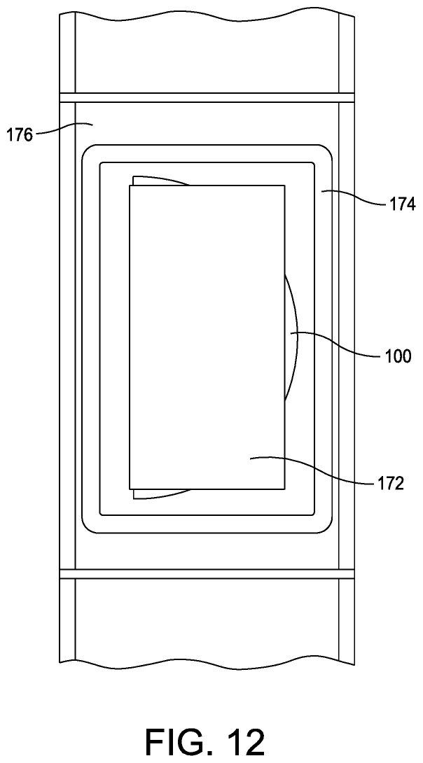

In one embodiment shown in FIG. 10, two pre-shaped, meshed ADM grafts 100 may be sutured together to form an ADM graft pocket 160. In this embodiment, the two pre-shaped, meshed ADM grafts 100 may be sutured together around the curved portions each of the semi-circular tissue portions 102, such that a breast implant 162 may be disposed within the ADM graft pocket 160 between the two pre-shaped, meshed ADM grafts 100. The implant 162 is thus supported from the bottom, without the need to be covered at the top. In one embodiment, the ADM graft pocket 160 may be pre-sutured and then packaged and stored for later surgical use, as discussed below in relation to FIGS. 11-12, or the ADM graft pocket may be formed from two pre-shaped, meshed ADM grafts 100 and sutured by the surgeon prior to or during a surgical procedure. In another operative embodiment applicable to reconstructive surgery, the implant may be wrapped in the pre-shaped, meshed ADM graft 100 from an anterior side, and the graft 100 sutured to the chest wall.

After manufacture and to provide complete a shelf-stable, packaged ADM graft product 170, the pre-shaped, meshed ADM graft 100 (or the ADM graft pocket 160) may be packaged along with two opposing pieces of backing material 172 and sterile water in a sealed medical sterilization pouch 174 such as, for example, a Kapak pouch (manufactured by AMPAK Technology Inc. of Larchmont, N.Y.), as shown in FIG. 11, or further into a sealed, peelable medical sterilization pouch 176 known as a “peel pouch” or a “chevron pouch,” as shown in FIG. 12. The packaged ADM graft product 170 may then be irradiated to a sterility assurance level (SAL) of 10⁻⁶ such that it may be stored at room temperature for up to two years. The packaged ADM graft product 170 may be labeled in any appropriate manner and may include information pertaining to the raw material, the shape, a use by date, special requirements, results of a visual inspection, and so on.

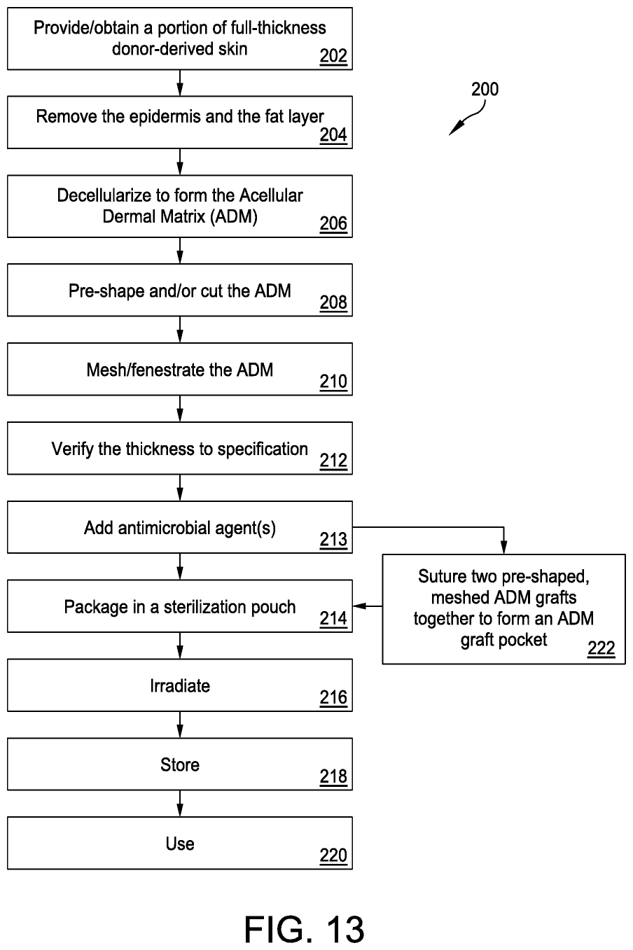

FIG. 13 provides a flowchart depicting an exemplary method (200) of manufacturing an embodiment of the pre-shaped, meshed ADM graft 100, the ADM graft pocket 160, and the packaged ADM graft product 170, discussed above. In this embodiment, the method may initiate with providing a portion of full-thickness donor-derived skin (202). Next, the epidermis layer and the fat layer adjacent to the dermis may be removed (204), and the dermal tissue may be decellularized according to a well-known or a proprietary decellularization process, resulting in the Acellular Dermal Matrix (ADM) (206). The ADM may then be shaped and/or cut into a pre-defined shape, such as the semi-circular tissue portion 102 or another appropriate shape, as necessary for an associated or pre-determined/assigned surgical procedure (208). The shaping may be accomplished using any appropriate scoring tool 130 or another appropriate shaping tool, and the graft may be cut out with the cutting tool.

The ADM may also be meshed/fenestrated in the desired mesh pattern (e.g., 1:1 graft:space ratio, 2:1 graft:space ratio) using any appropriate skin mesher 140 (210). The meshing or fenestrating process (210) may occur before or after the ADM is shaped into the pre-defined shape. The resulting pre-shaped, meshed ADM graft 100 may then be verified for its thickness to specification (e.g., 1 mm-2 mm) (212) using a thickness gauge, and one or more antimicrobial agents may be added to the pre-shaped, meshed ADM graft 100 to aid in post-surgical infection prevention (213). The graft 100 may then be packaged (214) between opposing pieces of backing material 172 within sterile water inside a self-sealing medical sterilization pouch 174 and/or a peelable pouch 176 such as, for example, a Kapak peel-pouch, forming the pre-shaped, meshed ADM graft product 170. The packaged ADM graft product 170 may be irradiated to SAL 10⁻⁶ (216). After irradiation (216), the packaged, pre-shaped, meshed ADM graft product 170 may be stored up to two years (218) before it is used in a surgical procedure (220).

In one embodiment, prior to packaging (214), two of the pre-shaped, meshed ADM grafts 100 may be joined (e.g., sutured) together about a curving portion of each individual graft 100 to form the ADM graft pocket 160 (222), discussed above in relation to FIG. 10. Alternatively, the ADM graft pocket 160 may be formed prior to a surgical procedure, within or prior to entering the operating theater.

The method of manufacturing the packaged, pre-shaped, meshed ADM graft product 170 provides a repeatable process for manufacturing the pre-shaped, meshed ADM graft 100 formed from full-thickness donor-derived skin such that surgeons may rely on the time-saving graft product in reconstructive surgical procedures to provide a graft solution that has the robust physical properties required of surgical skin grafts (as opposed to burn skin grafts), promotes healing in the form of effective drainage from the surgical site, and promotes integration of the graft into the patient's body.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

What is claimed is:

1. A method of manufacturing an acellular dermal matrix (ADM) graft product for use in a reconstructive surgical procedure, the method comprising:
providing a portion of donor-derived skin, the portion of the donor-derived skin having a full thickness;
removing an epidermis layer and a fat layer from the portion of the donor-derived skin to form a portion of dermal tissue;
decellularizing the portion of the dermal tissue to form a portion of ADM graft material;
forming the portion of the ADM graft material into a pre-defined shape in anticipation of the reconstructive surgical procedure;
fenestrating the pre-defined shape into a mesh pattern;
verifying that a thickness of the pre-defined shape equals a specified thickness;
packaging the pre-defined shape in a medical sterilization pouch to form a packaged, pre-shaped, and meshed ADM graft; and
irradiating the packaged, pre-shaped, and meshed ADM graft to a sterility assurance level of 10⁻⁶ to form the ADM graft product.

2. The method of claim 1, further comprising, prior to the packaging, adding one or more antimicrobial agents to the pre-defined shape.

3. The method of claim 1, wherein the specified thickness is between 1 mm and 2 mm.

4. The method of claim 1, wherein the forming the portion of the ADM graft material into the pre-defined shape comprises at least one of scoring and cutting the portion of the ADM graft material into a semi-circular shape having a notch configured to indicate a basement membrane surface of the semi-circular shape.

5. The method of claim 4, further comprising, prior to the packaging, joining two of the semi-circular shapes together along a curving portion of each of the two of the semi-circular shapes to form an ADM graft pocket configured to receive a breast implant.

6. The method of claim 1, wherein the forming the portion of the ADM graft material into the pre-defined shape comprises at least one of scoring and cutting the portion of the ADM graft material into an elliptical shape.

7. The method of claim 1, wherein the fenestrating the pre-defined shape comprises using a meshing tool to form a mesh pattern across an entirety of the pre-defined shape, the mesh pattern having an ADM tissue:space ratio of 1:1.

8. A pre-shaped, meshed acellular dermal matrix (ADM) graft stored as a packaged graft product prepared by a process comprising the steps of:
providing a portion of ADM tissue having a thickness between 1 mm and 2 fenestrating the portion of the ADM tissue in a mesh pattern extending over an entirety of the portion of the ADM tissue;
scoring the portion of the ADM tissue into a pre-defined shape to form the pre-shaped, meshed ADM graft;
verifying the thickness of the pre-shaped, meshed ADM graft;
packaging the pre-shaped, meshed ADM graft in a medical sterilization pouch; and
irradiating the pre-shaped, meshed ADM graft within the medical sterilization pouch to a sterility assurance level of 10⁻⁶ to form the packaged graft product.

9. The pre-shaped, meshed ADM graft stored as the packaged graft product prepared by the process of claim 8, wherein the pre-defined shape approximates a semi-circle having a notch that indicates a basement membrane surface of the pre-defined shape of the ADM tissue.

10. The pre-shaped, meshed ADM graft stored as the packaged graft product prepared by the process of claim 8, the process further comprising:
prior to the packaging, joining two of the pre-shaped, meshed ADM grafts to form an ADM graft pocket configured to receive a breast implant.

11. The pre-shaped, meshed ADM graft stored as the packaged graft product prepared by the process of claim 8, the process further comprising:
prior to the scoring, adding one or more antimicrobial agents to the portion of the ADM tissue.

12. The pre-shaped, meshed ADM graft stored as the packaged graft product prepared by the process of claim 8, wherein the mesh pattern has an ADM tissue:space ratio of 1:1.

13. The pre-shaped, meshed ADM graft stored as the packaged graft product prepared by the process of claim 8, wherein the packaged graft product has a shelf-life of two years.

14. An acellular dermal matrix (ADM) graft product, comprising:
an ADM graft derived from full-thickness skin, the ADM graft having a pre-formed shape with a mesh pattern formed therein; and
a medical sterilization pouch sealed about the ADM graft, wherein when the medical sterilization pouch and the ADM graft are irradiated to a sterility assurance level of 10⁻⁶, the ADM graft product has a shelf-life of two years.

15. The ADM graft product of claim 14, wherein the pre-formed shape comprises a semi-circular shape.

16. The ADM graft product of claim 15, wherein the semi-circular shape includes a notch that indicates a basement membrane surface of the ADM graft.

17. The ADM graft product of claim 14, wherein the mesh pattern extends across an entirety of the pre-formed shape, and wherein the mesh pattern has a material:space ratio of 1:1.

18. The ADM graft product of claim 14, wherein the mesh pattern extends across an entirety of the pre-formed shape, and wherein the mesh pattern has a material:space ratio of 2:1.

19. The ADM graft product of claim 14, the ADM graft having a thickness between 1 mm and 2 mm.

20. The ADM graft product of claim 14, wherein:
the pre-formed shape of the ADM graft includes a curved portion; and
the curved portion of the pre-formed shape is configured for attachment to an opposing curved portion of another pre-formed shape of another ADM graft to form an ADM graft pocket for receiving a breast implant.
